(19) United States
(12) Patent Application Publication
(10) Pub. No.: US 2023/0000397 A1
(43) Pub. Date: Jan. 5, 2023

(54) HEARING THRESHOLD AND/OR HEARING STATE DETECTION SYSTEM AND METHOD

(71) Applicants: HANGZHOU ERQINGCONG TECHNOLOGY CO., LTD, Hangzhou, Zhejiang (CN); TSINGHUA UNIVERSITY, Beijing (CN)

(72) Inventor: Qin GONG, Beijing (CN)

(21) Appl. No.: 17/782,972

(22) PCT Filed: May 13, 2020
(86) PCT No.: PCT/CN2020/089962

§ 371 Date: Jun. 6, 2022

(30) Foreign Application Priority Data
Dec. 31, 2019 (CN) 201911405753.X

(51) Int. Cl. A61B 5/12; A61B 5/00

(52) U.S. Cl. CPC A61B 5/125; A61B 5/7264; A61B 5/6817; A61B 5/7275; A61B 2562/0204

(57) ABSTRACT

Disclosure is a hearing threshold and/or hearing state detection system and method. The system comprises: an acquisition and transmission system configured to transmit stimulation signals and acquire an ear canal signal; and a hearing threshold analysis and prediction system including a hearing threshold detection module, a routine testing module and/or a hearing state screening module, wherein the hearing threshold detection module determines hearing thresholds at different stimulation frequencies through a pre-trained network model; the routine testing module adaptively selects a range of test intensities through the acquisition and transmission system, and predicts hearing thresholds related to different stimulation frequencies through a pre-trained network model; and the screening module is configured to perform hearing state screening through the acquisition and transmission system and a pre-trained network model. A detection result thereof is not only accurate, but also is applicable to various demand scenarios.

### RELATED APPLICATIONS

The present application is a U.S. National Phase of International Application Number PCT/CN2020/089962, filed May 13, 2022, and claims priority to Chinese Application Number 201911405753.X, filed Dec. 31, 2019.

### FIELD OF THE INVENTION

The present disclosure relates to a hearing threshold and/or hearing state detection system and method based on an I/O function of SFOAEs, which relates to the technical field of auditory system detection.

### BACKGROUND OF THE INVENTION

Otoacoustic Emissions (OAEs) is a kind of weak audio energy generated in the cochlea of the inner ear, transmitted through the ossicular chain and the tympanic membrane, and released to the external auditory canal, and is a part of normal function of the human ear. According to presence or absence of external acoustic stimuli, OAEs can be divided into two categories: Spontaneous Otoacoustic Emissions (SOAEs) and Evoked Otoacoustic Emissions (EOAEs). EOAEs are divided into three classes in terms of different evoked acoustic stimuli, i.e., Transient-Evoked Otoacoustic Emissions (TEOAEs), Distortion-Product Otoacoustic Emissions (DPOAEs) and Stimulus-Frequency Otoacoustic Emissions (SFOAEs).

Since a pure-tone hearing threshold test currently used in clinical practice is a kind of behavioral test, subjective feedback from a subject is required during the test, but it is greatly affected by subjective factors such as attention and degree of cooperation. Especially for those who are lack of cooperation (e.g., infants and young children), this detection method that requires the subjective feedback from the subject is not applicable. The SFOAEs refers to active emission of a weak sound signal at the same frequency as stimulus sound after the inner ear cochlea is subjected to stimulation of a single-frequency signal. SFOAEs can reflect active mechanism of cochlear outer hair cells so as to further reflect the function of the peripheral auditory system. Since the frequency of SFOAEs is exactly the same as that of the stimulus sound, SFOAEs have very good frequency specificity. Besides, since SFOAEs can be detected in moderately and severely deaf ears in the case that moderate or high stimulation intensity is applied, SFOAEs have a potential to objectively and quantitatively reflect a hearing threshold, and is especially suitable for hearing detection of those being lack of cooperation.

In the prior art, a portable full-featured otoacoustic emission detection system, specifically, a portable otoacoustic emission detection system based on a USB multimedia sound card has been disclosed, which realizes full-featured quantitative detection and analysis on signals of TEOAEs and DPOAEs. However, it neither involves detection of input-output (I/O) function of SFOAEs, nor a detection technology and method of using the I/O function of SFOAEs for hearing threshold estimation and hearing state screening of the auditory system. Further, an invention titled a Stimulus-Frequency Otoacoustic Emissions tuning curve detection and calibration system is also disclosed in the prior art, which only provides detection technology regarding a detection method and calibration system of SFOAEs' suppression tuning curve, but fails to involve a detection technology and method for hearing threshold estimation by using the I/O function of SFOAEs. Further disclosed in the prior art is an auditory sensitivity detection system based on SFOAEs, which provides detection of intensity and sensitivity by using waveform shapes of respective points of SFOAEs and detection of intensity and sensitivity by using waveform shapes of respective points of SFOAEs' suppression tuning curve, but fails to involve methods using, for example, an I/O function curve of SFOAEs.

To sum up, some of the existing technologies detect no hearing thresholds, while the other detect hearing thresholds without using complete information of the I/O output function of SFOAEs, as a result of which accuracy of hearing threshold detection results are not high.

### SUMMARY OF THE INVENTION

In view of the above problems, an objective of the present disclosure is to provide a hearing threshold or hearing state detection system and method capable of extracting a hearing threshold or a hearing state at a set frequency point in a rapidly and accurate manner.

In order to achieve the above objective, the present disclosure adopts the following technical schemes.

In a first aspect, the present disclosure provides a hearing threshold and/or hearing state detection system, comprising:

an acquisition and transmission system, configured to transmit stimulation signals and acquire an ear canal signal; and a hearing threshold analysis and prediction system, including a hearing threshold detection module, a routine testing module and/or a hearing state screening module.

The hearing threshold detection module inputs a preset range of stimulation frequencies through the acquisition and transmission system, and forms an I/O function curve at a detected frequency by detecting SFOAEs data resulted from all stimulation intensities at respective stimulation frequencies, extracts parameters of SFOAEs signals resulted from all stimulation intensities at each stimulation frequency, and predicts hearing thresholds at different stimulation frequencies through a pre-trained network model;

The routine testing module adaptively selects a range of test intensities through the acquisition and transmission system, forms an I/O function curve resulted within the range of test intensities at a detected frequency by detecting SFOAEs data resulted from all stimulation intensities at respective stimulation frequencies, extracts parameters of SFOAEs signals resulted from adaptively selected stimulation intensities at each stimulation frequency, and predicts hearing thresholds related to different stimulation frequencies through a pre-trained network model; and

The screening module is configured to input N preset stimulation intensities at a certain stimulation frequency through the acquisition and transmission system, acquire SFOAEs data resulted from each stimulation intensity, extract parameters of SFOAEs signals resulted from each stimulation intensity, and perform hearing state screening through a pre-trained network model.

Preferably, the acquisition and transmission system includes:

a signal sending device, configured to cause a stimulation signal source to send a digital signal;

a signal conversion device, configured to perform D/A or A/D conversion on a transmitted or received signal;

a stimulation signal delivering structure, configured to transmitting a stimulation signal to the human ear; and

a signal recovery structure, configured to acquire an ear canal signal.

Preferably, each of the hearing threshold detection module, the routine testing module and/or the hearing state screening module includes:

a stimulus sound parameter setting module, configured to set parameters of stimulus sound;

a suppression sound parameter setting module, configured to set parameters of suppression sound;

a stimulus sound signal generation module, configured to generate a corresponding digital stimulus sound signal according to the set parameters of stimulus sound;

a suppression sound signal generation module, configured to generate a corresponding digital suppression sound signal according to the set parameters of suppression sound;

a stimulus sound signal stimulation module, configured to send out a stimulus sound signal; and

a suppression sound signal stimulation module, configured to send out a suppression sound signal.

Preferably, the hearing threshold detection module further includes: a hearing threshold signal detection and processing module, configured to process the acquired ear canal signal, extract the SFOAEs signals resulted from of all stimulation intensities at different stimulation frequencies, and form the I/O function curve of SFOAEs, wherein the abscissa of the I/O function curve is set to stimulation intensity, and the ordinate is set to SFOAEs intensity;

a hearing threshold characteristic parameter extraction and principal component analysis module, configured to extract characteristic parameters and principal components of the I/O function curve of SFOAEs; and

a hearing threshold prediction module, configured to predict a hearing threshold at each stimulation frequency through the pre-trained network model according to the characteristic parameters and the principal components of the SFOAEs data resulted from all stimulation intensities at different stimulation frequencies, specifically including that:

if, at a certain stimulation frequency, a SFOAEs signal has been evoked by one within the preset range of all stimulation intensities, then the extracted characteristic parameters and the principal components are input into a pre-trained first network model to determine a hearing threshold related to the stimulation frequency; wherein the characteristic parameters and the principal components include: a first stimulation intensity that evokes the SFOAEs signal, recovery intensity, attenuation coefficient, and a maximum principal component obtained among signal-to-noise ratios of the SFOAEs signals resulted from all stimulation intensities; and

if, at a certain stimulation frequency, no SFOAEs signal is evoked by any within the preset range of all stimulation intensities, then the extracted characteristic parameters and the principal components are input into a pre-trained second network model to determine a hearing threshold related to the stimulation frequency; wherein the characteristic parameters and the principal components include: a maximum principal component among SFOAEs signal intensities resulted from all stimulation intensities, a maximum principal component among attenuation coefficients resulted from all stimulation intensities, and a maximum principal component among signal-to-noise ratios resulted from all stimulation intensities.

Preferably, the routine testing module further includes:

a routine test signal detection and processing module, configured to process the acquired ear canal signal, extract the SFOAE signals resulted from of the adaptively selected stimulation intensities at different stimulation frequencies, and form the I/O function curve of the SFOAEs resulted within the selected range of stimulation intensities, wherein the abscissa of the I/O function curve is set to stimulation intensity, and the ordinate is set to SFOAEs intensity;

a routine test characteristic parameter extraction and principal component analysis module, configured to extract characteristic parameters and principal components of the I/O function curve of the SFOAEs resulted from of the adaptively selected stimulation intensities; and a routine test prediction module, configured to stop signal acquisition upon data of the first stimulation intensity that can evoke SFOAEs and its subsequent consecutive M stimulation intensities has been detected at each stimulation frequency for acquiring, extract the characteristic parameters and the principal components of the SFOAEs data resulted within the range of stimulation intensities at this stimulation frequency, and predict a hearing threshold related to the stimulation frequency through a pre-trained network model, specifically including that:

if, at a certain stimulation frequency, a first SFOAEs signal has been evoked by one within the adaptively selected range of stimulation intensities, then the extracted characteristic parameters and the principal components are input into a pre-trained third network model to determine a hearing threshold related to the stimulation frequency; wherein the characteristic parameters include: a first stimulation intensity that evokes the SFOAEs signal, recovery intensity, attenuation coefficient, and a maximum principal component obtained among signal-to-noise ratios of the SFOAEs signals resulted from M+1 consecutive stimulation intensities; and if, at a certain stimulation frequency, no SFOAEs signal is evoked by any stimulation intensity within the adaptively selected range of stimulation intensities, then the extracted characteristic parameters and the principal components are input into a pre-trained second network model to determine a hearing threshold related to the stimulation frequency; wherein the characteristic parameters and the principal components include: a maximum principal component among SFOAEs signal intensities resulted within the adaptively selected range of stimulation intensities, a maximum principal component among attenuation coefficients resulted within the adaptively selected range of stimulation intensities, and a maximum principal component among signal-to-noise ratios resulted within the adaptively selected range of stimulation intensities.

Preferably, the screening module further includes:

a screening-related signal detection and processing module, configured to preprocess the ear canal signal, and extract the SFOAEs signals resulted from N specific stimulation intensities at a certain stimulation frequency;

a screening-related characteristic parameter extraction module, configured to extract characteristic parameters of the SFOAEs data; and

a screening-related prediction module, configured to predict a hearing state at the stimulation frequency through a pre-trained network model by using the characteristic parameters of the SFOAEs data resulted from the N specific stimulation intensities at the stimulation frequency, specifically including that:

the extracted characteristic parameters of the SFOAEs data are input into a pre-trained fourth network model to perform hearing state screening, wherein the characteristic parameters include N sets of characteristic parameters that are extracted separately from the SFOAEs data resulted from the N specific stimulation intensities at the stimulation frequency, and each set of characteristic parameters include: amplitude, signal-to-noise ratio, recovery intensity, attenuation coefficient, and signal-to-baseline ratio of the SFOAEs.

Preferably, the network models each adopt a network model constructed based on a machine learning algorithm or a network model constructed based on a multivariable statistical method;

wherein, the network model constructed based on the machine learning algorithm includes a support vector machine, a K-nearest neighbor, a BP neural network, a random forest and/or a decision tree neural network model; and

the network model constructed based on the multivariable statistical method include a network models based on discriminant analysis or based on logistic regression.

Preferably, the stimulation signal delivering structure includes an earphone amplifier and a micro loudspeaker that are connected in sequence;

The headphone amplifier is connected to an output end of the signal conversion structure, and the micro loudspeaker includes two electro-acoustic transducers for transmitting stimulus sound and suppression sound, respectively, so as to evoke a SFOAEs signal, the two electro-acoustic transducers are inserted into an earplug via two acoustic tubes, respectively, and input ends of the two electro-acoustic transducers are respectively connected to the headphone amplifier through two TRS interfaces, and the micro loudspeaker is configured to electro-acoustically convert an analog voltage signal into an acoustic signal which is sent to the ear of a subject via the earplug.

Preferably, the signal recovery structure includes a mini microphone and a microphone amplifier which are connected in sequence;

The mini microphone includes an acoustic-electric transducer, an input end of the mini microphone is inserted into the earplug through a transmission acoustic tube, an output end of the mini microphone is connected to an input end of the microphone amplifier, and an output end of the microphone amplifier is connected to an input end of the signal conversion structure.

In a second aspect, the present disclosure further provides a hearing threshold and/or hearing state detection method, comprising steps of:

S1, selecting a detection mode that a subject to be detected needs to undergo, wherein the detection mode is hearing threshold prediction, routine hearing threshold prediction or hearing state screening; wherein,

the hearing threshold prediction is configured to input a preset range of stimulation frequencies, form an I/O function curve at a detected frequency by detecting SFOAEs data resulted from all stimulation intensities at respective stimulation frequencies, extract parameters of SFOAEs signals resulted from all stimulation intensities at different stimulation frequencies, and determine hearing thresholds at different stimulation frequencies through a pre-trained network mode;

the routine hearing threshold prediction is configured to adaptively select a range of test intensities, form an I/O function curve resulted within the range of test intensities at a detected frequency by detecting SFOAEs data resulted from the adaptively selected stimulation intensities at respective stimulation frequencies, extract parameters of SFOAEs signals resulted from the adaptively selected stimulation intensities at each stimulation frequency, and determine a hearing threshold related to the stimulation frequency through a pre-trained network model; and

the hearing state screening is configured to input N preset stimulation intensities at a certain stimulation frequency, acquire SFOAEs data resulted from each stimulation intensity, extract parameters of SFOAEs signals resulted from each stimulation intensity, and perform hearing state screening through a pre-trained network model; and

S2, receiving, based on the selected detection mode and by the ear canal of a subject to be detected, different stimulation signals, and processing the ear canal signal to complete the hearing threshold prediction or the hearing state screening.

Further, when the detection mode selected by the subject to be detected is the hearing threshold prediction, a specific process thereof includes:

setting parameters of stimulus sound and suppression sound according to a specified range, and transmitting a stimulus sound signal and a suppression sound signal into an ear canal of the subject to be detected;

receiving an ear canal signal, and forming an I/O function curve at a detected frequency by detecting SFOAEs signals resulted from all stimulation intensities at respective stimulation frequencies, wherein the abscissa of the I/O function curve is set to be stimulus sound intensity, and the ordinate thereof is set to be SFOAEs intensity;

extracting characteristic parameters and principal components of the I/O function curve of the SFOAEs data; and

predicting a hearing threshold at each stimulation frequency through a pre-trained network model according to the characteristic parameters and the principal components of the SFOAEs data resulted from all stimulation intensities at different stimulation frequencies.

Further, predicting a hearing threshold at each stimulation frequency through a pre-trained network model according to the characteristic parameters and the principal components of the SFOAEs data resulted from all stimulation intensities at different stimulation frequencies, specifically includes that:

if, at a certain stimulation frequency, a SFOAEs signal has been evoked by one within the preset range of all stimulation intensities, then the extracted characteristic parameters and the principal components are input into a pre-trained first network model to determine a hearing threshold related to the stimulation frequency; wherein the characteristic parameters and the principal components include: a first stimulation intensity that evokes the SFOAEs signal, recovery intensity, attenuation coefficient, and a maximum principal component obtained among signal-to-noise ratios of the SFOAEs signals resulted from all stimulation intensities; and

if, at a certain stimulation frequency, no SFOAEs signal is evoked by any within the preset range of all stimulation intensities, then the extracted characteristic parameters and the principal components are input into a pre-trained second network model to determine a hearing threshold related to the stimulation frequency; wherein the characteristic parameters and the principal components include: a maximum principal component among SFOAEs signal intensities resulted from all stimulation intensities, a maximum principal component among attenuation coefficients resulted from all stimulation intensities, and a maximum principal component among signal-to-noise ratios resulted from all stimulation intensities.

Further, when the detection mode selected by the subject to be detected is the routine hearing threshold prediction, a specific process thereof includes:

adaptively selecting a range of test intensities, setting parameters of stimulus sound and suppression sound, and transmitting a stimulus sound signal and a suppression sound signal into the ear canal of the subject to be detected;

stopping signal acquisition upon data of a first stimulation intensity that can evoke SFOAEs and its subsequent consecutive M stimulation intensities has been detected, wherein M is a positive integer;

forming an I/O function curve resulted within the adaptively selected range of test intensities according to power spectrum signals of the SFOAEs resulted from different stimulation intensities at varied stimulation frequencies;

extracting characteristic parameters and principal components of the I/O function curves of the SFOAEs resulted within the adaptively selected range of test intensities; and

at each stimulation frequency for acquiring, stopping signal acquisition upon data of the first stimulation intensity that can evoke SFOAEs and its subsequent consecutive M stimulation intensities has been detected, extracting the characteristic parameters and the principal components of the SFOAEs data resulted within the adaptively selected range of stimulation intensities at this stimulation frequency, and predicting a hearing threshold related to the stimulation frequency through a pre-trained network model.

Further, predicting a hearing threshold related to the stimulation frequency through a pre-trained network model, specifically includes that:

if, at a certain stimulation frequency, a first SFOAEs signal has been evoked by one within the adaptively selected range of stimulation intensities, then the extracted characteristic parameters and the principal components are input into a pre-trained third network model to determine a hearing threshold related to the stimulation frequency; wherein the characteristic parameters include: a first stimulation intensity that evokes the SFOAEs signal, recovery intensity, attenuation coefficient, and a maximum principal component obtained among signal-to-noise ratios of the SFOAEs signals resulted from M+1 consecutive stimulation intensities; and if, at a certain stimulation frequency, no SFOAEs signal is evoked by any within the adaptively selected range of stimulation intensities, then the extracted characteristic parameters and the principal components are input into a pre-trained second network model to determine a hearing threshold related to the stimulation frequency; wherein the characteristic parameters and the principal components include: a maximum principal component among SFOAEs signal intensities resulted within the adaptively selected range of stimulation intensities, a maximum principal component among attenuation coefficients resulted within the adaptively selected range of stimulation intensities, and a maximum principal component among signal-to-noise ratios resulted within the adaptively selected range of stimulation intensities.

Further, when the detection mode selected by the subject to be detected is the hearing state screening, a specific process thereof includes:

setting parameters of stimulus sound and suppression sound, inputting specified N specific stimulation intensities at a certain stimulation frequency, and transmitting stimulus sound and suppression sound into the ear canal of the subject to be detected; extracting SFOAEs data signals resulted from the N specific stimulation intensities;

extracting characteristic parameters of the SFOAEs data; and

performing hearing state screening through a pre-trained fourth network model by using the extracted characteristic parameters of the SFOAEs resulted from N specific stimulation intensities at the stimulation frequency, wherein the characteristic parameters include N sets of characteristic parameters that are extracted separately from the SFOAEs data resulted from N specific stimulation intensities at the detected stimulation frequency, and each set of characteristic parameters include: amplitude, signal-to-noise ratio, recovery intensity, attenuation coefficient, and signal-to-baseline ratio of the SFOAEs.

In a third aspect, the present disclosure further provides a computer program including computer program instructions, wherein the program instructions, when being executed by a processor, are configured to implement the corresponding steps of the hearing threshold and hearing state detection method.

In a fourth aspect, the present disclosure further provides a storage medium on which computer program instructions are stored, wherein the program instructions, when being executed by a processor, are configured to implement the corresponding steps of the hearing threshold and hearing state detection method.

In a fifth aspect, the present disclosure further provides a terminal device including a processor and a memory, wherein the memory is configured to store at least one piece of executable instruction, and the executable instruction enables the processor to perform the corresponding steps of the hearing threshold and/or hearing state detection method.

The present disclosure has the following advantages by using the above technical solutions:

1. The present disclosure is provided based on an input/output function of SFOAEs, and can carry out various detection items according to a need of a subject to be detected, in which different stimulation frequencies and stimulation intensities are generated using the hearing threshold analysis and prediction system, and stimulation signals are send through the acquisition and transmission system, and then a signal in the ear canal of the subject to be detected is acquired and input to the hearing threshold analysis and prediction system for hearing threshold detection and/or hearing state screening, so that objective, rapid and accurate detection of a hearing threshold or a hearing state of the auditory system is realized;

2. The hearing threshold detection module of the present disclosure is configured to objectively and quantitatively extract a hearing threshold at a set frequency point, and can objectively detect a hearing threshold in clinic; the routine testing module performs an I/O function test based on adaptive selection of a range of test intensities to obtain a hearing threshold, and can enable rapid, objective and quantitative extraction of the hearing threshold at the set frequency point in clinic according to those required test intensities; and the screening module obtains a hearing state based on a specified number of specific stimulation intensities, and can enable rapid screening of hearing state according to rapid detection with the specified number of specific stimulation intensities;

In conclusion, the present disclosure can be widely applied in the field of auditory testing.

### BRIEF DESCRIPTION OF THE DRAWINGS

Various other advantages and benefits will become apparent to those of ordinary skill in the art by reading the detailed description of the preferred embodiments below. The drawings are only for the purpose of illustrating preferred embodiments, and should not be considered as limitation to the invention. The same reference numbers are used to refer to the same parts throughout the drawings. In the figures:

FIG. 1 is a schematic diagram of a structure of an embodiment of an acquisition and transmission system according to Embodiment 1 of the present disclosure;

FIG. 2 is a schematic flowchart of hearing threshold detection and hearing state screening according to Embodiment 1 of the present disclosure;

FIG. 3 is a schematic diagram of an instance of hearing threshold detection based on a hearing threshold detection module according to Embodiment 1 of the present disclosure;

FIG. 4 is a schematic flowchart of hearing threshold prediction performed by on a machine-learning-based network model during a hearing threshold testing process according to Embodiment 1 of the present disclosure;

FIG. 5 is a schematic flowchart of hearing threshold prediction performed by on a machine-learning-based network model during a routine hearing threshold testing process according to Embodiment 1 of the present disclosure;

FIG. 6 is a schematic flowchart of hearing state screening performed by on a machine-learning-based network model during a hearing state screening process according to Embodiment 1 of the present disclosure; and

FIGS. 7A and 7B are schematic diagrams of a first network model and a second network model according to Embodiment 1 of the present disclosure.

### DETAILED DESCRIPTION OF THE EMBODIMENTS

Exemplary embodiments of the present disclosure will be described in more detail below with reference to the accompanying drawings. While exemplary embodiments of the present disclosure are shown in the drawings, it should be understood that the present disclosure may be embodied in various forms and should not be limited by the embodiments set forth herein. Rather, these embodiments are provided for more thoroughly understanding the present disclosure and fully conveying the scope of the present disclosure to those skilled in the art.

It is to be understood that the terminology used herein is for a purpose of describing particular exemplary embodiments only and is not intended to construct limitation thereto. The singular forms “a,” “an,” and “the” as used herein can also be intended to include the plural forms unless the context dictates clearly otherwise. The wordings “comprise”, “include”, “contain” and “have” are inclusive, and are intended to indicate the presence of stated features, steps, operations, elements and/or components thereby, but not to exclude the presence or addition of one or multiple other features, steps, operations, elements, components, and/or combinations thereof. Method steps, processes, and operations described herein should not be construed as imposing them to be performed in a particular order as described or illustrated, unless a performed order has been explicitly indicated. It should be further understood that additional or alternative steps may also be used.

### Embodiment 1

A hearing threshold and/or hearing state detection system provided in the present embodiment detects a hearing threshold or a hearing state based on an I/O function of SFOAEs, including:

an acquisition and transmission system, which is configured to transmit a stimulation signal and acquire an ear canal signal; and

a hearing threshold analysis and prediction system, which is configured to perform signal analysis and processing to complete hearing threshold prediction or hearing state screening.

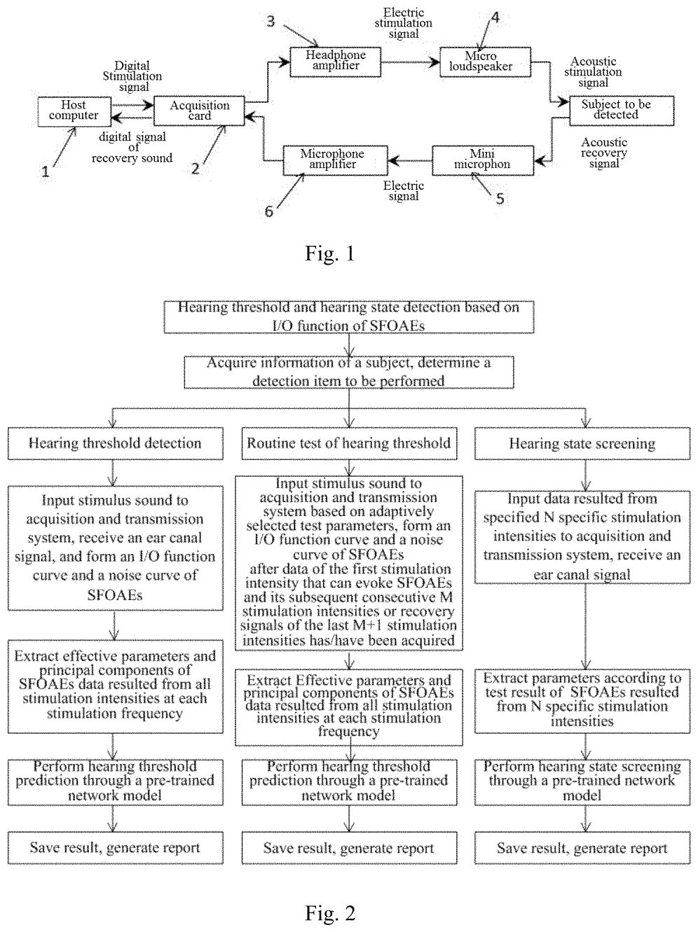

Specifically, as shown in FIG. 1, the acquisition and transmission system includes a signal sending device, a signal conversion device, a stimulation signal delivering structure, and a signal recovery structure.

The signal sending device is configured to send out a digital signal from a stimulation signal source, preferably, the signal sending device may use a computer 1 to send out the digital signal.

The signal conversion device is configured to perform A/D and D/A conversion on the signal. Preferably, the signal sending device may use an acquisition card 2 to implement the signal conversion. The acquisition card 2 adopts an acquisition card that is able to couple to the computer 1 so that the digital signal sent by the computer 1 is converted into an analog voltage signal. Preferably, a portable acquisition card with a sampling depth of 24 bits and a maximum sampling rate of 192 kHz may be used during detection, and is coupled to the computer 1 through a USB interface. Apparently, the signal conversion structure may also adopt other structures and other connection manners, for example, the acquisition card 2 is coupled to the computer 1 through an IEEE1394 interface, which will not be described any further here.

The stimulation signal delivering structure is configured to transmit a stimulation signal to the ear. Preferably, the stimulation signal delivering structure may include a headphone amplifier 3 and a micro loudspeaker 4 which are connected in sequence. The headphone amplifier 3 is connected to two output ends of the acquisition card 2 to implement power amplification and impedance matching for two output signals of the acquisition card 2. The micro loudspeaker 4 includes two electro-acoustic transducers that respectively generate stimulus sound and suppression sound for evoking a SFOAEs signal. The two electro-acoustic transducers are separately inserted into an earplug through two acoustic tubes, and input ends of these two electro-acoustic transducers are respectively connected to the headphone amplifier 3 through interfaces. The micro loudspeaker 4 is configured to electro-acoustically convert the analog voltage signal into an acoustic signal which is sent to the ear of a subject via the earplug. The micro loudspeaker 4 may adopt various products that can meet performance indexes requirement, such as a plug-in micro loudspeaker, etc., which is not limited here.

The signal recovery structure is configured to acquire back an otoacoustic emission signal and other signals in the external auditory canal of the ear. Preferably, the signal recovery structure includes a mini microphone 5 and a microphone amplifier 6 which are connected in sequence. In order to isolate the sound in the external auditory canal of the subject from the environment sound, in this embodiment, the micro loudspeaker 4 and the mini microphone 5 may be inserted into the same soft earplug. In this case, the mini microphone 5 includes an acoustic-electric transducer, which is configured to acquire the otoacoustic emission signal and other signals in the external auditory canal of the ear and convert the acquired acoustic signals into an electrical signal. An input end of the mini microphone 5 is inserted into the earplug through an acoustic tube, and the acoustic signals in the ear canal reach the acoustic-electric transducer via the acoustic tube and are converted into an analog voltage signal. An output end of the mini microphone 5 is connected to an input end of the microphone amplifier 6, and an output end of the microphone amplifier 6 is connected to an A/D input end of the acquisition card 2. The micro microphone 5 may adopt various products that can meet performance indexes requirement, such as a plug-in micro microphone, etc., which is not limited here. The microphone amplifier 6 is configured to amplify a signal output by the mini microphone 5. An amplification factor thereof may be adjusted according to actual requirements, and the adjusted factor includes, but is not limited to, 0 dB, 20 dB and 40 dB.

Specifically, an acquisition card drive system may further be provided in the computer 1. The acquisition card drive system is configured to drive a D/A port of the acquisition card 2 to receive a signal sent by the computer 1 and sent it to the subject's ear via the micro loudspeaker 4 after the signal is subjected to power amplification and impedance matching by the headphone amplifier 3; at the same time, an A/D port of the acquisition card 2 receives a signal sent back by the microphone amplifier 6 and sends it to the hearing threshold analysis and prediction system.

As shown in FIG. 2, the hearing threshold analysis and prediction system, upon performing hearing threshold estimation or hearing state screening, is configured to, first, obtain information of a subject to be detected, determine a detection item, and then activate varied testing modules according to different detection items. The hearing threshold analysis and prediction system includes a hearing threshold detection module based on the I/O function of SFOAEs, a routine testing module that performs an I/O function routine test based on adaptive selection of a range of test intensities, and a screening module which obtains a hearing state based on N specific intensities.

The hearing threshold detection module is configured to detect a hearing threshold of the subject to be detected, specifically, including: inputting different stimulation frequencies within a specified range through the acquisition and transmission system, and forming an I/O function curve and a noise curve of SFOAEs according to a recovery signal acquired under different stimulation intensities by the acquisition and transmission system; then, extracting characteristic parameters and principal components of SFOAEs data resulted from all stimulation intensities at each stimulation frequency, and determining a hearing threshold that corresponds to a related frequency point through a pre-trained network model.

The routine testing module detects a hearing threshold of the subject to be detected in a routine manner, specifically, including: inputting different stimulation intensities based on adaptive selection of a range of test intensities through the acquisition and transmission system, stopping acquiring signals upon data of the first stimulation intensity that can evoke SFOAEs and its subsequent consecutive M stimulation intensities or data of the last M+1 stimulation intensities has been acquired, and extracting characteristic parameters and principal components and inputting them into a pre-trained network model to predict a hearing threshold related to this stimulation frequency point. In this case, the routine testing module can realize rapid detection of the hearing threshold of the subject to be detected; M is a positive integer, and is specified according to specific situation of the subject to be detected and the accuracy of detection result. In this embodiment, a value of M may be 3, which is taken as an example but is not limited thereto. That is, in the case that a hearing threshold of the subject to be detected is detected in a routine manner by the routine testing module, at least data of four or more stimulation intensities are acquired.

The screening module screens a hearing state of the subject to be detected, specifically, including: inputting, under a certain stimulation frequency, N specific stimulation intensities through the acquisition and transmission system, and, predicting the hearing state through a pre-trained network model according to a test result of the SFOAEs resulted from a specific stimulation intensity and extracted characteristic parameters to complete screening of the hearing state of the subject to be detected. In this case, N is a positive integer, and is set according to the specific situation of the subject to be detected and the accuracy requirement of screening. In the embodiment, N may take a value of 3, that is, in the case that the screening module is activated to screen the hearing state of the subject to be detected, data of a total of three specified specific intensities are acquired, which is just taken as an example and is not limited thereto.

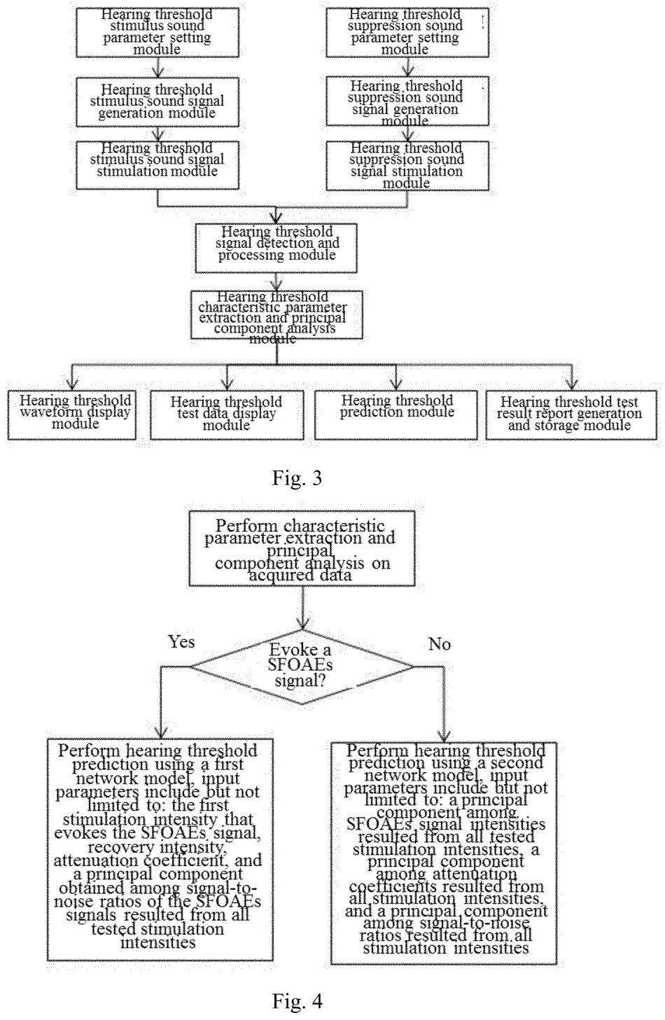

Specifically, as shown in FIG. 3 , the hearing threshold detection module includes a hearing threshold stimulus sound parameter setting module, a hearing threshold suppression sound parameter setting module, a hearing threshold stimulus sound signal generation module, a hearing threshold suppression sound signal generation module, a hearing threshold stimulus sound signal stimulation module, a hearing threshold suppression sound signal stimulation module, a hearing threshold signal detection and processing module, a hearing threshold characteristic parameter extraction and principal component analysis module, a hearing threshold waveform display module, a hearing threshold test data display module, a hearing threshold prediction module, and a hearing threshold test result report generation and storage module.

The hearing threshold stimulus sound parameter setting module is configured to set parameters of stimulus sound, such as a frequency of the stimulus sound, an intensity and a change step size of the stimulus sound, etc.;

The hearing threshold suppression sound parameter setting module is configured to set parameters of suppression sound, such as a frequency and an intensity of the suppression sound, etc.;

The hearing threshold stimulus sound signal generating module is configured to generate a corresponding digital stimulus sound signal according to the set parameters of stimulus sound, and send a corresponding signal to the hearing threshold stimulus sound signal stimulation module to send stimulus sound therefrom;

The hearing threshold suppression sound signal generation module is configured to generate a corresponding digital suppression sound signal according to the set parameters of suppression sound, and send a corresponding signal to the hearing threshold suppression sound signal stimulation module to send suppression sound therefrom;

The hearing threshold signal detection and processing module performs processing, such as coherent averaging, filtering on the acquired signals from the ear canal and then extracts power spectrum signals of the SFOAEs resulted from different stimulation intensities at varied stimulation frequencies, and then forms an input/output (I/O) function curve of the SFOAEs, which describes a relationship between the input stimulus sound intensities (abscissa) and the output SFOAEs intensities (ordinate). During specific detection, the hearing threshold stimulus sound signal stimulation module and the hearing threshold suppression sound signal stimulation module send out a stimulus sound signal and a suppression sound signal, respectively, to subject them to D/A conversion through the signal conversion structure and then deliver them to the subject's ear through the stimulation signal delivering structure; the signal recovery structure acquires and amplifies a signal returned from the external auditory canal of the subject, and sends it to the signal conversion structure; and the signal conversion structure performs A/D conversion on the signal and then sent it to the hearing threshold signal detection and processing module;

The hearing threshold characteristic parameter extraction and principal component analysis module is configured to extract characteristic parameters and principal components of the I/O function curve of SFOAEs. A characteristic parameter refers to that one extracted from the I/O function curve of SFOAEs and having a strong correlation with the hearing threshold. A principal component relates to transforming a set of potentially correlated original variables into an equal number of linearly uncorrelated variables through orthogonal transformation and further applying a model training method on them to extract the most correlated one to the hearing threshold, and then inputting the one into the hearing threshold prediction module.

The hearing threshold waveform display module dynamically displays power spectrum waveforms, baseline, and noise waveforms of the SFOAEs resulted from different stimulation intensities at varied stimulation frequencies, as well as the I/O function curve and a noise curve of the SFOAEs resulted from different stimulation frequencies, so that a detected state and a final result of the subject can be observed in real time. In this case, the noise waveform curve is used to observe whether the subject complies with the test requirements (a quiet state is required during the test);

The hearing threshold prediction module predicts the hearing thresholds under different stimulation frequencies by extracting the characteristic parameters and the principal components of the SFOAEs data resulted from all stimulation intensities at varied stimulation frequencies and subjecting them to a pre-trained network model; and

The hearing threshold test result report generation and storage module is configured to display detection data resulted from different stimulation intensities at varied frequencies, and generate and save all test results and test information of the subj ect.

As shown in FIGS. 3 and 4, in the case that the subject to be detected undergoes a detailed hearing threshold detection, the hearing threshold detection module is activated, in which a specific process is as following.

The parameters of the stimulus sound and the suppression sound are set according to a range specified in the hearing threshold detection module (for example, a stimulation frequency is set to a certain frequency in a range of 500 Hz-8 kHz), and a stimulus sound signal and a suppression sound signal are transmitted to the acquisition and transmission system; after receiving a recovery signal output by the acquisition and transmission system, the hearing threshold detection module forms an I/O function curve at the detected frequency by detecting the SFOAEs data resulted from all stimulation intensities at respective stimulation frequency points; and then the hearing threshold characteristic parameter extraction and principal component analysis module extracts corresponding characteristic parameters and principal components for analysis. The extracted characteristic parameters include: stimulation intensity, and amplitude, signal-to-noise ratio, recovery intensity, attenuation coefficient and the like of the SFOAEs resulted from different stimulation intensities. A principal component refers to the largest one that is extracted among data of all signal-to-noise ratios resulted from all stimulation intensities. Then, the hearing threshold prediction module performs hearing threshold prediction, a specific process of which is as following.

If, at a certain stimulation frequency, a SFOAEs signal has been evoked by one within the range of stimulation intensities, then the extracted characteristic parameters and the principal components are input into a pre-trained machine-learning-based first network model to determine a hearing threshold related to the corresponding stimulation frequency point, so that the hearing threshold prediction is performed. The characteristic parameters and the principal components input into the first network model include, but are not limited to: the first stimulation intensity that evokes the SFOAEs signal, recovery intensity, attenuation coefficient, and the maximum principal component obtained among signal-to-noise ratios of the SFOAEs signals resulted from all tested stimulation intensities. In this case, a specific approach for acquiring the maximum principal component obtained among the signal-to-noise ratios of the SFOAEs signals includes that: for example, under a certain stimulation frequency subjected to a range of stimulation intensities of 5 dB-70 dB, data of a total of 14 stimulation intensities are acquired, and 14 signal-to-noise ratios of an input/output (I/O) function curve of SFOAEs are extracted; the 14 signal-to-noise ratios are subjected to a principal component analysis (PCA) method to extract out 14 principal components that are orthogonal to each other, and then the largest two principal components are selected; then, through a set of training, the one with the greatest correlation with a pure tone hearing threshold is extracted from the two largest principal components to sever as an input parameter of the network model. In this embodiment, the principal component with the greatest correlation with the pure tone hearing threshold is exactly the larger of the largest two principal components. In addition, the other three characteristic parameters input into the first network model (i.e., the first stimulation intensity that evokes the SFOAEs signal, recovery intensity, and attenuation coefficient) are also in the set of training. Many characteristic parameters extracted from the I/O function curve of SFOAEs are subjected to correlation analysis against the pure tone hearing threshold, and three characteristic parameters with the greatest correlation thereto are extracted. As such, an input layer of the machine-learning-based first network model has a total of 4 parameters, i.e., the first stimulation intensity that evokes the SFOAEs signal, recovery intensity, attenuation coefficient, and the maximum principal component obtained among the signal-to-noise ratios of the SFOAEs signals resulted from all tested stimulation intensities. This above is taken as an example, and the methods for obtaining a principal component of a characteristic parameter in other models are similar thereto, which will not be described any further;

If, at a certain stimulation frequency, no SFOAEs signal is evoked by any within the range of stimulation intensities, then a trained machine-learning-based second network model is used to perform hearing threshold prediction. Parameters input into the second network model include, but are not limited to: the maximum principal component among SFOAEs signal intensities resulted from all tested stimulation intensities, the maximum principal component among attenuation coefficients resulted from all stimulation intensities, and the maximum principal component among signal-to-noise ratios resulted from all stimulation intensities.

Specifically, the routine testing module includes a routine test stimulus sound parameter setting module, a routine test suppression sound parameter setting module, a routine test stimulus sound signal generation module, a routine test suppression sound signal generation module, a routine test stimulus sound signal stimulation module, a routine test suppression sound signal stimulation module, a routine test signal detection and processing module, a routine test characteristic parameter extraction and principal component analysis module, a routine test waveform display module, a routine test data display module, a routine test prediction module, and a routine test result report generation and storage module.

The routine test stimulus sound parameter setting module is configured to set parameters of stimulus sound, such as a frequency of the stimulus sound, an initial intensity of the stimulus sound, an intensity change step size of the stimulus sound, etc.;

The routine test suppression sound parameter setting module is configured to set parameters of suppression sound, such as a frequency and an intensity of the suppression sound;

The routine test stimulus sound signal generation module and the routine test suppression sound signal generation module, respectively, generate a corresponding digital stimulus sound signal and a corresponding digital suppression sound signal according to the set parameters, and send the corresponding signals to the routine test stimulus sound signal stimulation module and the routine test suppression sound signal stimulation module;

The routine test signal detection and processing module performs processing, such as coherent averaging, filtering on the acquired signals and then extracts power spectrum signals of the SFOAEs resulted from different stimulation intensities at varied stimulation frequencies, and finally forms an I/O function curve resulted within the range of test intensities. During detection, the routine test stimulus sound signal stimulation module and the routine test suppression sound signal stimulation module send out a stimulus sound signal and a suppression sound signal, respectively, to subject them to D/A conversion through the signal conversion structure and then deliver them to the subject's ear through the stimulation signal delivering structure; the signal recovery structure acquires and amplifies a signal returned from the external auditory canal of the subject, and sends it to the signal conversion structure; and the signal conversion structure performs A/D conversion on the signal and then sent it to the hearing threshold signal detection and processing module;

The routine test characteristic parameter extraction and principal component analysis module is configured to extract characteristic parameters and principal components of the I/O function curve of SFOAEs;

The routine test waveform display module dynamically displays detection data (including amplitude, baseline, phase and noise of power spectrum) of the SFOAEs resulted from different stimulation intensities at varied stimulation frequencies, as well as amplitude values and related noise of the I/O function of the SFOAEs resulted from different stimulation intensities at varied stimulation frequencies;

The routine test prediction module stops acquiring signals upon data of the first stimulation intensity that can evoke SFOAEs and its subsequent consecutive M stimulation intensities or data of the last M+1 stimulation intensities has been acquired, and extracts the characteristic parameters and the principal components and inputs them into a pre-trained machine-learning-based network model to predict a hearing threshold corresponding to this stimulation frequency point; and

The routine test result report generation and storage module is configured to generate and save all test results and test information of the subject.

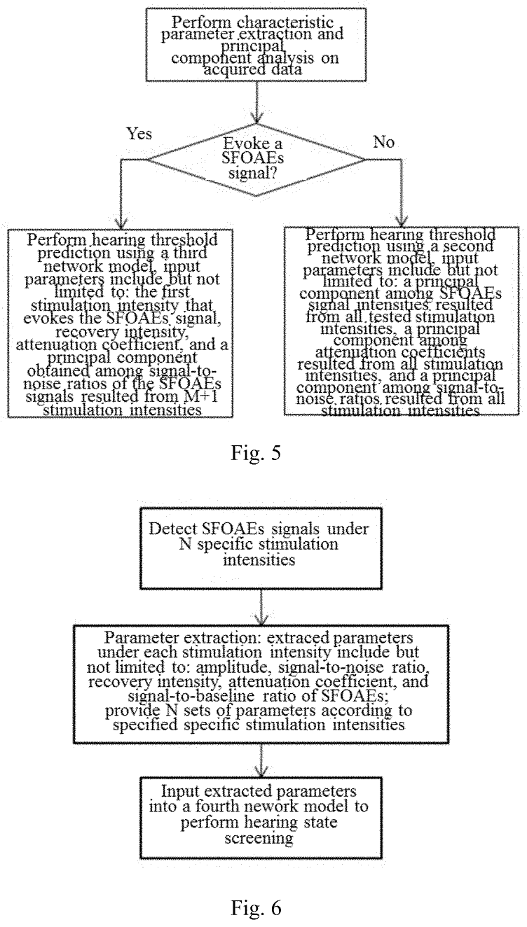

As shown in FIG. 5, in the case that the subject to be detected needs to undergo routine testing of hearing threshold, a specific calculation process after activation of the routine testing module is as following.

A stimulation frequency is configured to increase in octaves in a range of 500 Hz-8 kHz. The routine testing module selects a range of test intensities in an adaptive manner, sets parameters of stimulus sound and suppression sound, inputs initial stimulation intensities in an adaptive and random manner under different stimulation frequencies, and transmits a stimulus sound signal and a suppression sound signal to the acquisition and transmission system; upon detecting data of the first stimulation intensity that can evoke SFOAEs and its consecutive M stimulation intensities or data of the last M+1 stimulation intensities, signal acquisition is stopped; a recovery signal output by the acquisition and transmission system is input into the routine testing module, and an I/O function curve resulted within the range of test intensities is formed according to the power spectrum signals of the SFOAEs resulted from different stimulation intensities at varied stimulation frequencies; the routine test characteristic parameter extraction and principal component analysis module in the routine testing module extracts corresponding characteristic parameters and performs analysis. The extracted characteristic parameters including: stimulation intensity, and amplitude, signal-to-noise ratio, recovery intensity, attenuation coefficient and the like of the SFOAEs subjected to different stimulation intensities. In the present embodiment, M takes a value of 3, that is, the hearing threshold of the subject to be detected is detected in a routine manner by using data from at least 4 stimulation intensities in a routine test. Then, according to extraction and analysis results of the routine test characteristic parameter extraction and principal component analysis module, hearing threshold prediction is performed through the routine test prediction module, specifically including that:

If a SFOAEs signal has been evoked by one within the range of stimulation intensities, then the extracted characteristic parameters are input into a trained machine-learning-based third network model to predict a hearing threshold related to this frequency point. In this case, the characteristic parameters input to the third network model include, but are not limited to: the first stimulation intensity that evokes the SFOAEs signal, recovery intensity, attenuation coefficient, and the maximum principal component obtained among signal-to-noise ratios resulted from four consecutive stimulation intensities; and

If no SFOAEs signal is evoked by any within the range of stimulation intensities, then the pre-trained second neural network model is used to perform hearing threshold prediction. Parameters input into the second network model include, but are not limited to: the maximum principal component among extracted SFOAEs signal intensities resulted from all tested stimulation intensities, the maximum principal component among extracted attenuation coefficients resulted from all stimulation intensities, and the maximum principal component among extracted signal-to-noise ratios resulted from all stimulation intensities.

Specifically, the screening module is configured to perform hearing state screening through a pre-trained machine-learning-based network model, and includes a screening-related stimulus sound parameter setting module, a screening-related suppression sound parameter setting module, and a screening-related stimulus sound signal generation module, a screening-related suppression sound signal generation module, a screening-related stimulus sound signal stimulation module, a screening-related suppression sound signal stimulation module, a screening-related signal detection and processing module, a screening-related characteristic parameter extraction module, a screening-related waveform display module, and a screening-related test data display module for screening out I/O of SFOAEs resulted from a specific stimulation intensity, in which,

The screening-related stimulus sound parameter setting module is configured to set parameters of stimulus sound, such as a frequency of the stimulus sound;

The screening-related suppression sound parameter setting module is configured to set parameters of suppression sound, such as a frequency and an intensity of the suppression sound;

The screening-related stimulus sound signal generation module and the screening-related suppression sound signal generation module generate a corresponding digital stimulus sound signal and a corresponding digital suppression sound signal, respectively, according to the set parameters, and send the corresponding signals to the screening-related stimulus sound signal stimulation module and the screening-related suppression sound signal stimulation module;

The screening-related signal detection and processing module performs processing, such as coherent averaging, filtering on the acquired signals and then extracts power spectrum signals of the SFOAEs resulted from N specific stimulation intensities at a certain stimulation frequency (in this embodiment, N takes a value of 3, and the specific stimulation intensities at the certain stimulation frequency may include three groups: 55 dB, 60 dB, 65 dB). During detection, the screening-related stimulus sound signal stimulation module and the screening-related suppression sound signal stimulation module send out a stimulus sound signal and a suppression sound signal, respectively, to subject them to D/A conversion through the signal conversion structure and then deliver them to the subject's ear through the stimulation signal delivering structure; the signal recovery structure acquires and amplifies a signal returned from the external auditory canal of the subject, and then sends it to the signal conversion structure; and the signal conversion structure performs A/D conversion on the signal and then sent it to the screening-related signal detection and processing module;

The screening-related characteristic parameter extraction module is configured to extract characteristic parameters of the SFOAEs data. The characteristic parameters include: amplitude, signal-to-noise ratio, recovery intensity, attenuation coefficient, and signal-to-baseline ratio of the SFOAEs;

The screening-related test data display module dynamically displays detection data of the SFOAEs resulted from different stimulation intensities at varied stimulation frequencies;

The screening-related prediction module extracts 5*N effective characteristic parameters by extracting the characteristic parameters of the SFOAEs resulted from 3 specific stimulation intensities at this stimulation frequency, and predicts a hearing state related to this stimulation frequency point through a pre-trained machine-learning-based network model; and

The screening-related test result report generation and storage module is configured to generate and save all test results and test information of the subject.

In the case that the subject to be detected needs to undergo hearing state screening, a specific calculation process after activation of the screening module is as following.

Under a certain stimulation frequency, specified N specific stimulation intensities are input by the screening module, a stimulus sound signal and a suppression sound signal are transmitted to the acquisition and transmission system, and a feedback signal output by the acquisition and transmission system is input into the screening module; the screening-related signal detection and processing module in the screening module extracts power spectrum signals of the SFOAEs resulted from the N specific stimulation intensities, as shown in FIG. 6, and sends them to the screening-related characteristic parameter extraction module to extract the desired characteristic parameters which include, but are not limited to: amplitude, signal-to-noise ratio, recovery intensity, attenuation coefficient, and signal-to-baseline ratio of the SFOAEs; and the extracted characteristic parameters are input into a trained machine-learning-based fourth network model to perform hearing state screening. The parameters input to the fourth network model are three groups of characteristic parameters that are extracted separately according to the SFOAEs data resulted from 3 specific stimulation intensities at the detected frequency, each group of characteristic parameters including, but is not limited to: amplitude, signal-to-noise ratio, recovery intensity, attenuation coefficient, signal-to-baseline ratio of the SFOAEs.

In some embodiments of the present disclosure, the first network model is configured to predict a hearing threshold; the second network model is configured to predict a hearing threshold; the third network model is configured to predict a hearing threshold; and the fourth network model is configured to screen a hearing state. The first, second, third, and fourth network models may adopt a network model constructed based on a machine learning algorithm or a network model constructed based on a multivariable statistical method. The first, second, third, and fourth network models are pre-built and trained, respectively, and are preset in the hearing threshold analysis and prediction system or the hearing state screening system, respectively. The network models constructed based on the multivariable statistical methods include network models based on discriminant analysis or logistic regression. The network models constructed based on the machine learning algorithms include network models such as support vector machines, K-nearest neighbors, BP neural networks, random forests, and decision trees. In this case, a process of predicting a hearing threshold using the machine-learning-based first or second network model is briefly described below, which is taken as an example but not limited thereto, and specifically including that:

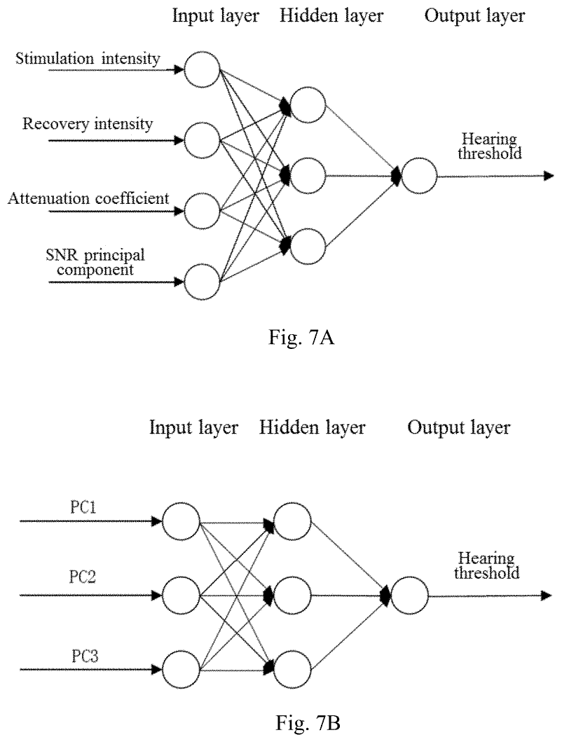

In the present embodiment, both the first network model and the second network model use a machine-learning-based BP neural network (Back-propagation network, BPNN) model. The BP neural network model is a feedforward neural network, which uses a supervised learning technique called backpropagation for training. As shown in FIGS. 7A and 7B, the diagram FIG. 7A is the first network model, and the diagram FIG. 7B is the second network model. The BP neural network used in this embodiment is a three-layer network including an input layer, a hidden layer and an output layer. The number of nodes in the input layer is set to be the number of input variables of the model. The number of nodes in an input layer of the first network model in this embodiment is set to 4, and the parameters of the nodes in the input layer are, respectively, the first stimulation intensity that evokes a SFOAEs signal, recovery intensity, attenuation coefficient, and the maximum principal component obtained among signal-to-noise ratios of the SFOAEs signals resulted from all tested stimulation intensities (represented by “SNR principal component” in the figure); and the number of nodes in an input layer of the second network model in this embodiment is set to 3, and the parameters of the input layer nodes are, respectively, the maximum principal component among SFOAEs signal intensities resulted from all test stimulation intensities, the maximum principal component among attenuation coefficients resulted from all stimulation intensities, and the maximum principal component among signal-to-noise ratios resulted from all stimulation intensities, which are represented by principal component 1, principal component 2, and principal component 3 in the figure, respectively. In this embodiment, the number of nodes in an hidden layer is set to 3, and only one node is provided in an output layer of a BP neural network model for predicting the hearing threshold, that is, the predicted hearing threshold; and the number of nodes in an output layer of a BP neural network-based classification model (i.e., the fourth network model in this embodiment) is set to 2, that is, normal hearing or impaired hearing. Training of these BP neural network models is divided into forward propagation of an operation signal and back propagation of an error signal. Weights are continually updated so that an actual output is closer to an expected output, and the weights are fixed once the error signal is reduced to a set minimum value or an upper limit of the training steps has been reached.

### Embodiment 2

The present embodiment further provides a hearing threshold and/or hearing state detection method, including the following steps.

S1, A detection mode that a subject to be detected needs to undergo is selected, wherein the detection mode is hearing threshold prediction, routine hearing threshold prediction or hearing state screening;

S2, Based on the selected detection mode, the acquisition and transmission system transmits different stimulation signals to the subject to be detected and acquires an ear canal signal; and the hearing threshold analysis and prediction system processes the ear canal signal to complete the hearing threshold prediction or the hearing state screening.

In some embodiments of the present disclosure, in the case that the detection mode selected by the subject to be detected is the hearing threshold prediction, a specific process thereof includes: setting parameters of stimulus sound and suppression sound according to a specified range, and transmitting a stimulus sound signal and a suppression sound signal into the ear canal of the subject to be detected;

receiving an ear canal signal, and forming an I/O function curve at a detected frequency by detecting SFOAEs signals resulted from all stimulation intensities at respective stimulation frequency points, wherein the abscissa of the I/O function curve is set to be stimulus sound intensity, and the ordinate thereof is set to be SFOAEs intensity;

extracting characteristic parameters and principal components of the I/O function curve of the SFOAEs data; and predicting a hearing threshold at each stimulation frequency point through a pre-trained network model according to the characteristic parameters and the principal components of the SFOAEs data resulted from all stimulation intensities at different stimulation frequencies, a specific process of which includes the following.

If, at a certain stimulation frequency, a SFOAEs signal has been evoked by one within the preset range of stimulation intensities, then the extracted characteristic parameters and the principal components are input into a pre-trained machine-learning-based first network model to determine a hearing threshold related to the corresponding stimulation frequency point. The characteristic parameters and the principal components input into the first network model include, but are not limited to: the first stimulation intensity that evokes the SFOAEs signal, recovery intensity, attenuation coefficient, and the maximum principal component obtained among signal-to-noise ratios of the SFOAEs signals resulted from all stimulation intensities; and

If, at a certain stimulation frequency, no SFOAEs signal is evoked by any within the preset range of stimulation intensities, then the extracted characteristic parameters and the principal components are input into a pre-trained machine-learning-based second network model to determine a hearing threshold related to this stimulation frequency point. In this case, the characteristic parameters and the principal components include: the maximum principal component among SFOAEs signal intensities resulted from all stimulation intensities, the maximum principal component among attenuation coefficients resulted from all stimulation intensities, and the maximum principal component among signal-to-noise ratios resulted from all stimulation intensities.

In some embodiments of the present disclosure, in the case that the detection mode selected by the subject to be detected is the routine hearing threshold prediction, a specific process thereof includes:

adaptively selecting a range of test intensities, setting parameters of stimulus sound and suppression sound, and transmitting a stimulus sound signal and a suppression sound signal into the ear canal of the subject to be detected;

stopping signal acquisition upon data of the first stimulation intensity that can evoke SFOAEs and its subsequent consecutive M stimulation intensities has been detected, where M is a positive integer;

forming an I/O function curve of the SFOAEs resulted within the range of test intensities according to power spectrum signals of the SFOAEs resulted from different stimulation intensities at varied stimulation frequencies;

extracting characteristic parameters and principal components of the I/O function curves of the SFOAEs resulted within the range of test intensities; and

at each stimulation frequency for acquiring, stopping signal acquisition upon data of the first stimulation intensity that can evoke SFOAEs and its subsequent consecutive M stimulation intensities has been detected, extracting the characteristic parameters and the principal components of the SFOAEs data resulted within the range of stimulation intensities at this stimulation frequency, and predicting a hearing threshold related to the stimulation frequency point through a pre-trained network model, which specifically includes the following.

If, at a certain stimulation frequency, a SFOAEs signal has been evoked by one within the range of stimulation intensities, then the extracted characteristic parameters are input into a trained machine-learning-based third network model to predict a hearing threshold related to this frequency point. In this case, the characteristic parameters input to the third network model include, but are not limited to: the first stimulation intensity that evokes the SFOAEs signal, recovery intensity, attenuation coefficient, and the maximum principal component obtained among signal-to-noise ratios resulted from M+1 consecutive stimulation intensities; and

If, at a certain stimulation frequency, no SFOAEs signal is evoked by any within the range of stimulation intensities, then the extracted characteristic parameters and the principal components are input into a pre-trained machine-learning-based second network model to determine a hearing threshold related to this stimulation frequency point. In this case, the characteristic parameters and the principal components include: the maximum principal component among SFOAEs signal intensities resulted from all stimulation intensities, the maximum principal component among attenuation coefficients resulted from all stimulation intensities, and the maximum principal component among signal-to-noise ratios resulted from all stimulation intensities.

In some embodiments of the present disclosure, in the case that the detection mode selected by the subject to be detected is the hearing state screening, a specific process thereof includes:

setting parameters of stimulus sound and suppression sound, inputting specified N specific stimulation intensities at a certain stimulation frequency, and transmitting the stimulus sound and the suppression sound into the ear canal of the subject to be detected; extracting SFOAEs data signals resulted from the N specific stimulation intensities;

extracting characteristic parameters of the SFOAEs data; and performing hearing state screening through a pre-trained machine-learning-based fourth network model by using the extracted characteristic parameters of the SFOAEs resulted from the N specific stimulation intensities at this stimulation frequency. In this case, the characteristic parameters include N sets of characteristic parameters that are extracted separately from the SFOAEs data resulted from N specific stimulation intensities at the detected stimulation frequency, and each set of characteristic parameters include: amplitude, signal-to-noise ratio, recovery intensity, attenuation coefficient, and signal-to-baseline ratio of the SFOAEs.

### Embodiment 3

The present embodiment further provides a computer program including computer program instructions, wherein the program instructions, when being executed by a processor, are configured to implement the steps of the hearing threshold and hearing state detection method according to Embodiment 2.

### Embodiment 4

The present embodiment further provides a storage medium on which computer program instructions are stored, wherein the program instructions, when being executed by a processor, are configured to implement the steps of the hearing threshold and hearing state detection method according to Embodiment 2.

### Embodiment 5

The present embodiment further provides a terminal device including a processor and a memory, wherein the memory is configured to store at least one piece of executable instruction, and the executable instruction enables the processor to perform the steps of the hearing threshold and/or hearing state detection method according to Embodiment 2.

To sum up, the present disclosure is provided based on the input and output (I/O) function of SFOAEs, in which the I/O function curve of the SFOAEs resulted from different stimulation frequencies is utilized in conjunction with the principal component analysis, a pre-trained network model is adopted to perform hearing threshold detection, and the characteristic parameters of the SFOAEs signal resulted from specific intensities are utilized to perform hearing state screening through a pre-trained network model. The detection results thereof are accurate and applicable to various demand scenarios.

The above-mentioned embodiments are only used to illustrate the present disclosure, and the structure, connection method and manufacturing process of each component can be changed to some extent. Any equivalent transformation and improvement based on the technical solution of the present disclosure should not be excluded from the scope of protection of the present disclosure.

1. A hearing threshold and/or hearing state detection system, wherein, the system comprises:
an acquisition and transmission system, configured to transmit stimulation signals and acquire an ear canal signal; and
a hearing threshold analysis and prediction system, including a hearing threshold detection module, a routine testing module and/or a hearing state screening module, wherein
the hearing threshold detection module inputs a preset range of stimulation frequencies through the acquisition and transmission system, and forms an I/O function curve at a detected frequency by detecting Stimulus-Frequency Otoacoustic Emissions, SFOAEs, data resulted from all stimulation intensities at respective stimulation frequencies, extracts parameters of SFOAEs signals resulted from all stimulation intensities at each stimulation frequency, and predicts hearing thresholds at different stimulation frequencies through a pre-trained network model;
the routine testing module adaptively selects a range of test intensities through the acquisition and transmission system, forms an I/O function curve resulted within the range of stimulation intensities at a detected frequency by detecting SFOAEs data resulted from all stimulation intensities at respective stimulation frequencies, extracts parameters of SFOAEs signals resulted from adaptively selected stimulation intensities at each stimulation frequency, and predicts hearing thresholds related to different stimulation frequencies through a pre-trained network model; and
the screening module is configured to input N preset stimulation intensities at a certain stimulation frequency through the acquisition and transmission system, acquire SFOAEs data resulted from each stimulation intensity, extract parameters of SFOAEs signals resulted from each stimulation intensity, and perform hearing state screening through a pre-trained network model.

2. The hearing threshold and/or hearing state detection system according to claim 1, wherein, the acquisition and transmission system comprises:
a signal sending device, configured to cause a stimulation signal source to send a digital signal;
a signal conversion device, configured to perform D/A or A/D conversion on a transmitted or received signal;
a stimulation signal delivering structure, configured to transmitting a stimulation signal to a human ear; and
a signal recovery structure, configured to acquire an ear canal signal.

3. The hearing threshold and/or hearing state detection system according to claim 1, wherein, each of the hearing threshold detection module, the routine testing module and/or the hearing state screening module includes:
a stimulus sound parameter setting module, configured to set parameters of stimulus sound;
a suppression sound parameter setting module, configured to set parameters of suppression sound;
a stimulus sound signal generation module, configured to generate a corresponding digital stimulus sound signal according to the set parameters of stimulus sound;
a suppression sound signal generation module, configured to generate a corresponding digital suppression sound signal according to the set parameters of suppression sound;
a stimulus sound signal stimulation module, configured to send out a stimulus sound signal; and
a suppression sound signal stimulation module, configured to send out a suppression sound signal.

4. The hearing threshold and/or hearing state detection system according to claim 1 wherein, the hearing threshold detection module further includes:
a hearing threshold signal detection and processing module, configured to process the acquired ear canal signal, extract the SFOAEs signals resulted from of all stimulation intensities at different stimulation frequencies, and form the I/O function curve of SFOAEs, wherein an abscissa of the I/O function curve is set to stimulation intensity, and an ordinate is set to SFOAEs intensity;
a hearing threshold characteristic parameter extraction and principal component analysis module, configured to extract characteristic parameters and principal components of the I/O function curve of SFOAEs; and
a hearing threshold prediction module, configured to predict a hearing threshold at each stimulation frequency through the pre-trained network model according to the characteristic parameters and the principal components of the SFOAEs data resulted from all stimulation intensities at different stimulation frequencies, specifically including that:
if, at a certain stimulation frequency, a SFOAEs signal has been evoked by one within the preset range of all stimulation intensities, then the extracted characteristic parameters and the principal components are input into a pre-trained first network model to determine a hearing threshold related to the stimulation frequency; wherein the characteristic parameters and the principal components include: a first stimulation intensity that evokes the SFOAEs signal, recovery intensity, attenuation coefficient, and a maximum principal component obtained among signal-to-noise ratios of the SFOAEs signals resulted from all stimulation intensities; and
if, at a certain stimulation frequency, no SFOAEs signal is evoked by any within the preset range of all stimulation intensities, then the extracted characteristic parameters and the principal components are input into a pre-trained second network model to determine a hearing threshold related to the stimulation frequency; wherein the characteristic parameters and the principal components include: a maximum principal component among SFOAEs signal intensities resulted from all stimulation intensities, a maximum principal component among attenuation coefficients resulted from all stimulation intensities, and a maximum principal component among signal-to-noise ratios resulted from all stimulation intensities.

5. The hearing threshold and/or hearing state detection system according to claim 1, wherein, the routine testing module further includes:
a routine test signal detection and processing module, configured to process the acquired ear canal signal, extract the SFOAE signals resulted from of the adaptively selected stimulation intensities at different stimulation frequencies, and form the I/O function curve of SFOAEs resulted within the selected range of stimulation intensities, wherein an abscissa of the I/O function curve is set to stimulation intensity, and an ordinate is set to SFOAEs intensity;
a routine test characteristic parameter extraction and principal component analysis module, configured to extract characteristic parameters and principal components of the I/O function curve of SFOAEs resulted from of the adaptively selected stimulation intensities; and
a routine test prediction module, configured to stop signal acquisition upon data of the first stimulation intensity that can evoke SFOAEs and its subsequent consecutive M stimulation intensities has been detected at each stimulation frequency for acquiring, extract the characteristic parameters and the principal components of the SFOAEs data resulted within the range of stimulation intensities at this stimulation frequency, and predict a hearing threshold related to the stimulation frequency through a pre-trained network model, specifically including that:
if, at a certain stimulation frequency, a first SFOAEs signal has been evoked by one within the adaptively selected range of stimulation intensities, then the extracted characteristic parameters and the principal components are input into a pre-trained third network model to determine a hearing threshold related to the stimulation frequency; wherein the characteristic parameters include: a first stimulation intensity that evokes the SFOAEs signal, recovery intensity, attenuation coefficient, and a maximum principal component obtained among signal-to-noise ratios of the SFOAEs signals resulted from M+1 consecutive stimulation intensities; and
if, at a certain stimulation frequency, no SFOAEs signal is evoked by any within the adaptively selected range of stimulation intensities, then the extracted characteristic parameters and the principal components are input into a pre-trained second network model to determine a hearing threshold related to the stimulation frequency; wherein the characteristic parameters and the principal components include: a maximum principal component among SFOAEs signal intensities resulted within the adaptively selected range of stimulation intensities, a maximum principal component among attenuation coefficients resulted within the adaptively selected range of stimulation intensities, and a maximum principal component among signal-to-noise ratios resulted within the adaptively selected range of stimulation intensities.

6. The hearing threshold and/or hearing state detection system according to claim 1, wherein, the screening module further includes:
a screening-related signal detection and processing module, configured to preprocess the ear canal signal, and extract the SFOAEs signals resulted from N specific stimulation intensities at a certain stimulation frequency;
a screening-related characteristic parameter extraction module, configured to extract characteristic parameters of SFOAEs; and
a screening-related prediction module, configured to predict a hearing state at the stimulation frequency through a pre-trained network model by using the characteristic parameters of the SFOAEs data resulted from the N specific stimulation intensities at the stimulation frequency, specifically including that:
the extracted characteristic parameters of SFOAEs data are input into a pre-trained fourth network model to perform hearing state screening, wherein the characteristic parameters include N sets of characteristic parameters that are extracted separately from the SFOAEs data resulted from the N specific stimulation intensities at the stimulation frequency, and each set of characteristic parameters include: amplitude, signal-to-noise ratio, recovery intensity, attenuation coefficient, and signal-to-baseline ratio of SFOAEs.

7. The hearing threshold and/or hearing state detection system according to claim 1, wherein, the network models each adopt a network model constructed based on a machine learning algorithm or a network model constructed based on a multivariable statistical method;
wherein the network model constructed based on the machine learning algorithm includes a support vector machine, a K-nearest neighbor, a BP neural network, a random forest and/or a decision tree neural network model; and
the network model constructed based on the multivariable statistical method include a network models based on discriminant analysis or based on logistic regression.

8. The hearing threshold and/or hearing state detection system according to claim 2, wherein, the stimulation signal delivering structure includes an earphone amplifier and a micro loudspeaker that are connected in sequence;
the headphone amplifier is connected to an output end of the signal conversion structure, and the micro loudspeaker includes two electro-acoustic transducers for transmitting stimulus sound and suppression sound, respectively, so as to evoke a SFOAEs signal, the two electro-acoustic transducers are inserted into an earplug via two acoustic tubes, respectively, and input ends of the two electro-acoustic transducers are respectively connected to the headphone amplifier through two TRS interfaces, and the micro loudspeaker is configured to electro-acoustically convert an analog voltage signal into an acoustic signal which is sent to the ear of a subject via the earplug.

9. The hearing threshold and/or hearing state detection system according to claim 2, characterized in thatwherein, the signal recovery structure includes a mini microphone and a microphone amplifier which are connected in sequence;
the mini microphone includes an acoustic-electric transducer, an input end of the mini microphone is inserted into the earplug through a transmission acoustic tube, an output end of the mini microphone is connected to an input end of the microphone amplifier, and an output end of the microphone amplifier is connected to an input end of the signal conversion structure.

10. A hearing threshold and/or hearing state detection method, wherein, the method comprises steps of:
S1, selecting a detection mode that a subject to be detected needs to undergo, wherein the detection mode is hearing threshold prediction, routine hearing threshold prediction or hearing state screening; wherein,
the hearing threshold prediction is configured to input a preset range of stimulation frequencies, form an I/O function curve at a detected frequency by detecting SFOAEs data resulted from all stimulation intensities at respective stimulation frequencies, extract parameters of SFOAEs signals resulted from all stimulation intensities at different stimulation frequencies, and determine hearing thresholds at different stimulation frequencies through a pre-trained network mode;
the routine hearing threshold prediction is configured to adaptively select a range of test intensities, form an I/O function curve resulted within the range of test intensities at a detected frequency by detecting SFOAEs data resulted from the adaptively selected stimulation intensities at respective stimulation frequencies, extract parameters of SFOAEs signals resulted from the adaptively selected stimulation intensities at each stimulation frequency, and determine a hearing threshold related to the stimulation frequency through a pre-trained network model; and
the hearing state screening is configured to input N preset stimulation intensities at a certain stimulation frequency, acquire SFOAEs data resulted from each stimulation intensity, extract parameters of SFOAEs signals resulted from each stimulation intensity, and perform hearing state screening through a pre-trained network model; and
S2, receiving, based on the selected detection mode and by the ear canal of a subject to be detected, different stimulation signals, and processing the ear canal signal to complete the hearing threshold prediction or the hearing state screening.

11. The hearing threshold and/or hearing state detection method according to claim 10, wherein, when the detection mode selected by the subject to be detected is the hearing threshold prediction, a specific process thereof includes:
setting parameters of stimulus sound and suppression sound according to a specified range, and transmitting a stimulus sound signal and a suppression sound signal into an ear canal of the subject to be detected;
receiving an ear canal signal, and forming an I/O function curve at a detected frequency by detecting SFOAEs signals resulted from all stimulation intensities at respective stimulation frequencies, wherein an abscissa of the I/O function curve is set to be stimulus sound intensity, and an ordinate thereof is set to be SFOAEs intensity;
extracting characteristic parameters and principal components of the I/O function curve of SFOAEs; and
predicting a hearing threshold at each stimulation frequency through a pre-trained network model according to the characteristic parameters and the principal components of the SFOAEs data resulted from all stimulation intensities at different stimulation frequencies.

12. The hearing threshold and/or hearing state detection method according to claim 11, characterized in thatwherein, predicting a hearing threshold at each stimulation frequency through a pre-trained network model according to the characteristic parameters and the principal components of the SFOAEs data resulted from all stimulation intensities at different stimulation frequencies, specifically includes that:
if, at a certain stimulation frequency, a SFOAEs signal has been evoked by one within the preset range of all stimulation intensities, then the extracted characteristic parameters and the principal components are input into a pre-trained first network model to determine a hearing threshold related to the stimulation frequency; wherein the characteristic parameters and the principal components include: a first stimulation intensity that evokes the SFOAEs signal, recovery intensity, attenuation coefficient, and a maximum principal component obtained among signal-to-noise ratios of the SFOAEs signals resulted from all stimulation intensities; and
if, at a certain stimulation frequency, no SFOAEs signal is evoked by any within the preset range of all stimulation intensities, then the extracted characteristic parameters and the principal components are input into a pre-trained second network model to determine a hearing threshold related to the stimulation frequency; wherein the characteristic parameters and the principal components include: a maximum principal component among SFOAEs signal intensities resulted from all stimulation intensities, a maximum principal component among attenuation coefficients resulted from all stimulation intensities, and a maximum principal component among signal-to-noise ratios resulted from all stimulation intensities.

13. The hearing threshold and/or hearing state detection method according to claim 10, wherein, when the detection mode selected by the subject to be detected is the routine hearing threshold prediction, a specific process thereof includes:
adaptively selecting a range of test intensities, setting parameters of stimulus sound and suppression sound, and transmitting a stimulus sound signal and a suppression sound signal into an ear canal of the subject to be detected;
stopping signal acquisition upon data of a first stimulation intensity that can evoke SFOAEs and its subsequent consecutive M stimulation intensities has been detected, wherein M is a positive integer;
forming an I/O function curve resulted within the adaptively selected range of test intensities according to power spectrum signals of the SFOAEs data resulted from different stimulation intensities at varied stimulation frequencies;
extracting characteristic parameters and principal components of the I/O function curves of the SFOAEs data resulted within the adaptively selected range of test intensities; and
at each stimulation frequency for acquiring, stopping signal acquisition upon data of the first stimulation intensity that can evoke SFOAEs and its subsequent consecutive M stimulation intensities has been detected, extracting the characteristic parameters and the principal components of the SFOAEs data resulted within the adaptively selected range of stimulation intensities at this stimulation frequency, and predicting a hearing threshold related to the stimulation frequency through a pre-trained network model.

14. The hearing threshold and/or hearing state detection method according to claim 13, wherein, predicting a hearing threshold related to the stimulation frequency through a pre-trained network model, specifically includes:
if, at a certain stimulation frequency, a first SFOAEs signal has been evoked by one within the adaptively selected range of stimulation intensities, then the extracted characteristic parameters and the principal components are input into a pre-trained third network model to determine a hearing threshold related to the stimulation frequency; wherein the characteristic parameters include: a first stimulation intensity that evokes the SFOAEs signal, recovery intensity, attenuation coefficient, and a maximum principal component obtained among signal-to-noise ratios of the SFOAEs signals resulted from M+1 consecutive stimulation intensities; and
if, at a certain stimulation frequency, no SFOAEs signal is evoked by any within the adaptively selected range of stimulation intensities, then the extracted characteristic parameters and the principal components are input into a pre-trained second network model to determine a hearing threshold related to the stimulation frequency; wherein the characteristic parameters and the principal components include: a maximum principal component among SFOAEs signal intensities resulted within the adaptively selected range of stimulation intensities, a maximum principal component among attenuation coefficients resulted within the adaptively selected range of stimulation intensities, and a maximum principal component among signal-to-noise ratios resulted within the adaptively selected range of stimulation intensities.

15. The hearing threshold and/or hearing state detection method according to claim 10, wherein, when the detection mode selected by the subject to be detected is the hearing state screening, a specific process thereof includes:
setting parameters of stimulus sound and suppression sound, inputting specified N specific stimulation intensities at a certain stimulation frequency, and transmitting stimulus sound and suppression sound into an ear canal of the subject to be detected; extracting SFOAEs data signals resulted from the N specific stimulation intensities;
extracting characteristic parameters of SFOAEs; and
performing hearing state screening through a pre-trained fourth network model by using the extracted characteristic parameters of the SFOAEs data resulted from N specific stimulation intensities at the stimulation frequency, wherein the characteristic parameters include N sets of characteristic parameters that are extracted separately from the SFOAEs data resulted from N specific stimulation intensities at the detected stimulation frequency, and each set of characteristic parameters include: amplitude, signal-to-noise ratio, recovery intensity, attenuation coefficient, and signal-to-baseline ratio of SFOAEs.

16. A computer program comprising computer program instructions, wherein the program instructions, when being executed by a processor, are configured to implement the corresponding steps of the hearing threshold and hearing state detection method according to claim 10.

17. A storage medium on which computer program instructions are stored, wherein the program instructions, when being executed by a processor, are configured to implement the corresponding steps of the hearing threshold and hearing state detection method according to claim 10.

18. A terminal device comprising a processor and a memory, wherein the memory is configured to store at least one piece of executable instruction, and the executable instruction enables the processor to perform the corresponding steps of the hearing threshold and/or hearing state detection method according to claim 10.
